(19) United States
(12) Patent Application Publication
(10) Pub. No.: US 2012/0109267 A1
(43) Pub. Date: May 3, 2012

(54) HEATING SYSTEM FOR A HYPERTHERMIA APPARATUS

(75) Inventors: J. Michael Fausset, Lafayette, IN (US); Michael Sturdevant, Concord Township, OH (US); Edward Schneider, Eastlake, OH (US)

(73) Assignee: THERMALTHERAPEUTIC SYSTEMS, INC., Pittsburgh, PA (US)

(21) Appl. No.: 12/913,140

(22) Filed: Oct. 27, 2010

(51) Int. Cl. A61F 7/00

(52) U.S. Cl. 607/96

(57) ABSTRACT

A heating system for a hyperthermia apparatus may include a heater having a plurality of independently controllable heater elements located on one or more surfaces of the heater, and a temperature sensor associated with one or more of the heater elements. Each of the surfaces may be proximate to a reservoir cartridge. The heating system may include a computing device in communication with the temperature sensor. The computing device may be configured to adjust an amount of electrical current to the one or more heater elements associated with the temperature sensor based on a temperature reading received from the temperature sensor.

### CROSS REFERENCE TO RELATED APPLICATIONS

This application is related to U.S. patent application Ser. No. 12/054,013 filed Mar. 24, 2008, the entirety of which is hereby incorporated by reference.

### BACKGROUND

The use of a thermal therapy device to deliver intraperitoneal hyperthermia in conjunction with surgery and/or chemotherapy has resulted in positive survival and quality of life outcomes for patients who may have otherwise had only weeks or months to live. The dramatic response is due in part to direct contact of heat or medication with diseased areas. Intraperitoneal hyperthermia has proven a successful treatment for numerous ailments, including, but not limited to cancer. Exposing affected cells to heat, therapeutic agents and/or medication has a more aggressive and profound effect on patient outcomes.

Conventional hyperthermia apparatuses utilize a passive heating system, such as a heat exchanger, to heat a fluid to be supplied to a patient. A heat exchanger is typically connected to a water tank via a set of tubes. The water tank is commonly connected to a heater that heats water in the tank. Heated tank water is pumped to the heat exchanger, which typically has two compartments, one compartment containing water and a second compartment containing a fluid to be administered to a patient. The two compartments are typically separated by a metal plate. The heated water from the tank heats the water in the first compartment of the heat exchanger. The water in the heat exchanger then heats the metal plate which in turn heats the fluid. As such, the fluid is heated by a series of heat transfers and not by a direct heat transfer to the fluid.

This indirect approach to heating fluid results in heat loss due to the numerous points of heat exchange (i.e. from the tank to the heat exchanger, from one compartment of the heat exchanger to the metal plate, from the metal plate to the other compartment and from the heat exchanger to the patient). Moreover, conventional hyperthermia apparatuses are large and weigh in excess of three-hundred pounds. This limits mobility and storage capacity, and renders the apparatus unsuitable for use outside of operating rooms.

### SUMMARY

Before the present methods are described, it is to be understood that this invention is not limited to the particular systems, methodologies or protocols described, as these may vary. It is also to be understood that the terminology used herein is for the purpose of describing particular embodiments only, and is not intended to limit the scope of the present disclosure which will be limited only by the appended claims.

It must be noted that as used herein and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art. As used herein, the term “comprising” means “including, but not limited to.”

In an embodiment, a heating system for a hyperthermia apparatus may include a heater having a plurality of independently controllable heater elements located on one or more surfaces of the heater, and a temperature sensor associated with one or more of the heater elements. Each of the surfaces may be proximate to a reservoir cartridge. The heating system may include a computing device in communication with the temperature sensor. The computing device may be configured to adjust an amount of electrical current to the one or more heater elements associated with the temperature sensor based on a temperature reading received from the temperature sensor.

A heating system for a hyperthermia apparatus may include a heater that has a first portion having a first surface, a second portion having a second surface, a third portion having a third surface, a plurality of independently controllable heater elements located on one or more of the first surface, the second surface and the third surface, and a temperature sensor associated with one or more of the heater elements. The heating system may include a computing device in communication with the temperature sensor. The computing device may be configured to adjust an amount of electrical current to one or more heater elements associated with the temperature sensor based on a temperature reading received from the temperature sensor.

### BRIEF DESCRIPTION OF THE DRAWINGS

Embodiments of the invention are better understood with reference to the following drawings. The elements of the drawings are not necessarily to scale relative to each other. Like reference numerals designate corresponding similar parts.

Aspects, features, benefits and advantages of the present invention will be apparent with regard to the following description and accompanying drawings, of which:

FIG. 1 depicts exemplary elements of a hyperthermia apparatus according to an embodiment.

FIG. 2 depicts exemplary elements of a hyperthermia apparatus operating in prime mode according to an embodiment.

FIG. 3 depicts exemplary elements of a hyperthermia apparatus contained in a housing according to an embodiment.

FIG. 4 depicts an exemplary reservoir cartridge and screen according to an embodiment.

FIG. 5 depicts an exemplary pressure isolator according to an embodiment.

FIG. 6 depicts an exemplary heater according to an embodiment.

FIGS. 7A and 7B depict exemplary heaters according to an embodiment.

FIGS. 8A and 8B depict exemplary reservoir cartridges and heaters according to an embodiment.

### DETAILED DESCRIPTION

For purposes of the discussion below, the term “comprising” means “including, but not limited to.” As used herein, the term “about” means plus or minus 10% of the numerical value of the number with which it is being used. For example about 50% means in the range of 45%-55%.

A “therapeutic agent” means an agent utilized to treat, combat, ameliorate or prevent an unwanted condition or disease of a patient. In an embodiment, a therapeutic agent may include a chemotherapeutic agent.

“Administering” when used in conjunction with a therapeutic agent means to administer a therapeutic agent into or onto a target tissue or to administer a therapeutic agent to a patient whereby the therapeutic agent positively impacts the tissue to which it is targeted. “Administering” a composition may be accomplished by oral administration, injection, infusion or absorption or in conjunction with intraperitoneal hyperthermia or by a combination of such techniques. Such techniques may further include heating, radiation and ultrasound.

A “target” refers to the material for which either deactivation, rupture, disruption or destruction or preservation, maintenance, restoration or improvement of function or state is desired. For example, diseased cells, pathogens, or infectious material may be considered undesirable material in a diseased subject and may be a target for therapy.

The term “treating” may be taken to mean prophylaxis of a specific disorder, disease or condition, alleviation of the symptoms associated with a specific disorder, disease or condition and/or prevention of the symptoms associated with a specific disorder, disease or condition.

A “patient” generally refers to any living organism to which to compounds described herein are administered and may include, but is not limited to, any non-human mammal, primate or human. Such “patients” may or my not be exhibiting the signs, symptoms or pathology of the particular diseased state.

The terms “effective” or “therapeutically effective” as used herein may refer to eliciting a biological or medicinal response in a tissue, organ, system, animal, individual or human that is being sought by a researcher, veterinarian, medical doctor or other clinician. A biological or medicinal response may include, for example, one or more of the following: (1) inhibiting a disease, condition or disorder in an individual that is experiencing or displaying the pathology or symptoms of the disease, condition or disorder or arresting further development of the pathology and/or symptoms of the disease, condition or disorder, and (2) ameliorating a disease, condition or disorder in an individual that is experiencing or exhibiting the pathology or symptoms of the disease, condition or disorder or reversing the pathology and/or symptoms experienced or exhibited by the individual.

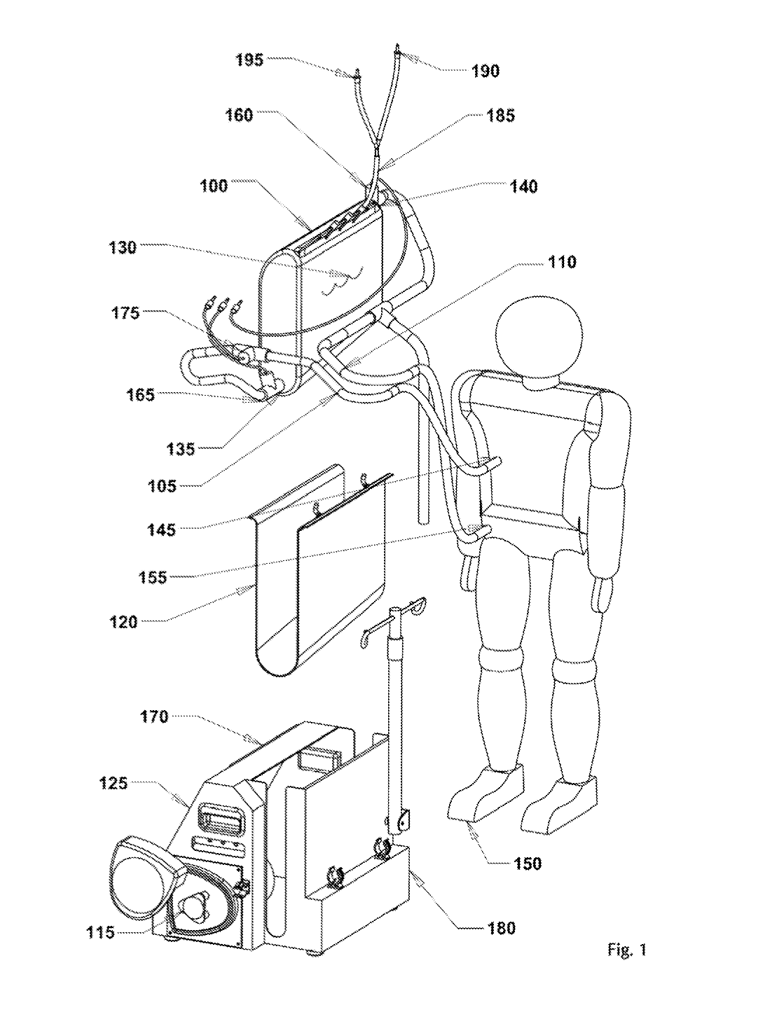

FIG. 1 illustrates an exemplary hyperthermia apparatus according to an embodiment. As illustrated by FIG. 1, a hyperthermia apparatus may include a reservoir cartridge 100, an outflow tube 105, an inflow tube 110, a pump 115, a heater 120, a computer 125 and a housing 180.

In an embodiment, a heater 120 may be in close proximity to reservoir cartridge 100 to maximize heat transfer to fluid contained within the reservoir cartridge. Heater 120 may be, for example, a thermoelectric heater for providing electric heat to fluid 130. A thermoelectric heater may facilitate direct heat transfer to fluid 130. In alternative embodiments, heater 120 is any type of heater known in the art, such as a water bath, immersion heater or the like.

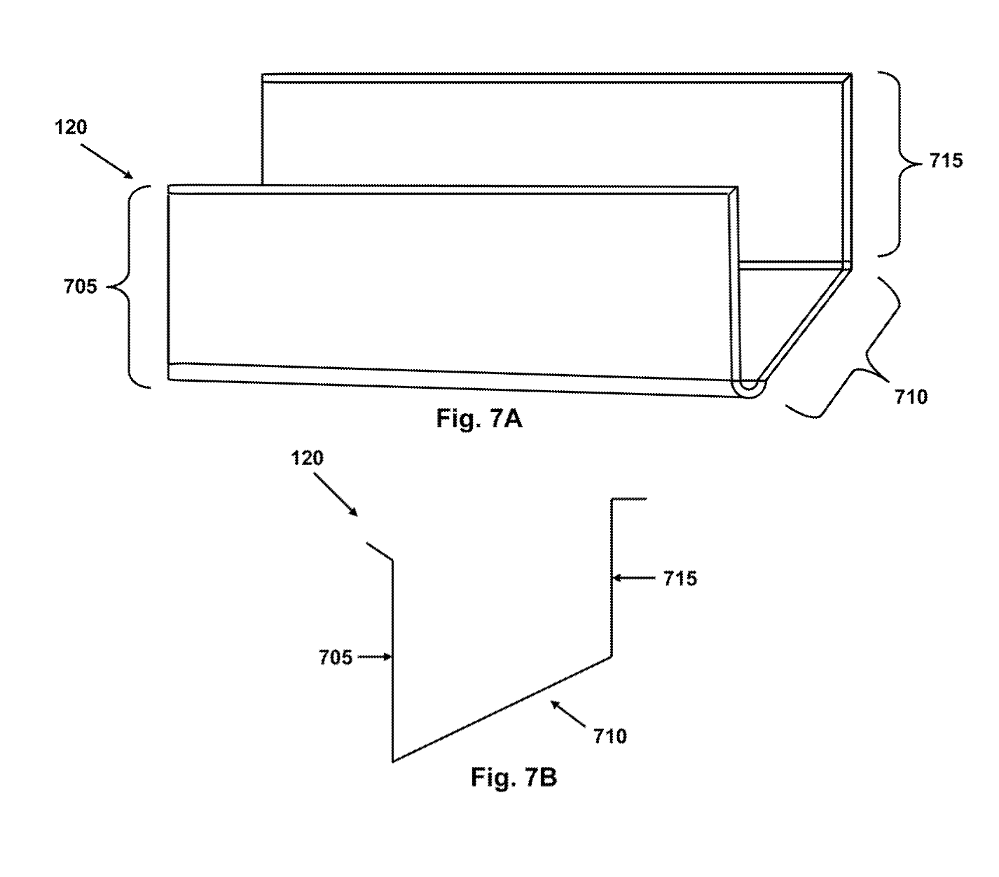

FIG. 7A illustrates an exemplary heater according to an embodiment. As illustrated by FIG. 7A, a heater 120 may have a first portion 705, a second portion 710 and a third portion 715. In an embodiment, the first portion 705 and third portion 715 may be connected to the second portion 710. The first portion 705 and the second portion 710 may be substantially parallel. In an embodiment, the second portion 710 may be angled relative to the first portion 705 and/or the third portion 715. For example, the second portion 710 may be positioned at approximately a 45 degree angle relative to the first portion 705 and/or the third portion 715. FIG. 7B illustrates a side view of the heater illustrated in FIG. 7A.

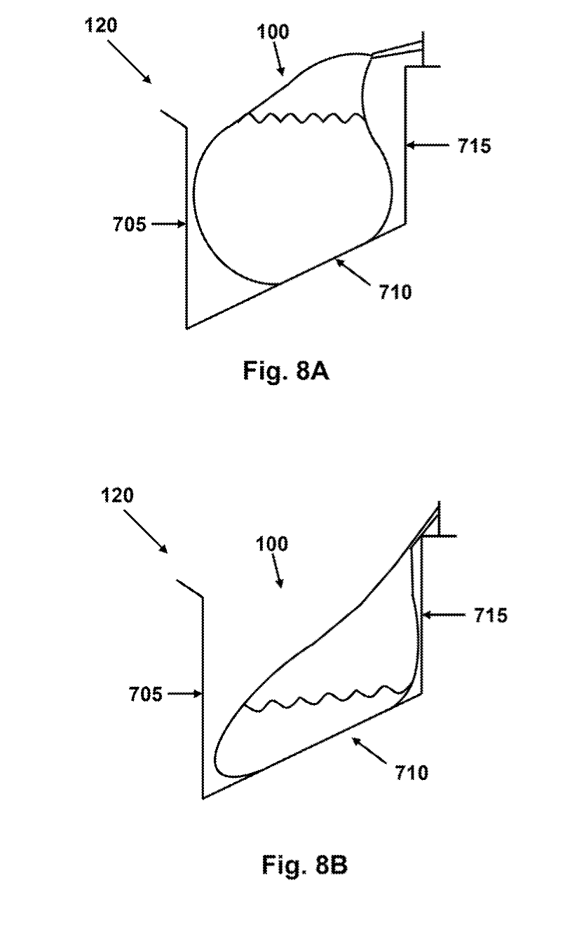

In an embodiment, the heater configuration illustrated in FIG. 7A and FIG. 7B may help provide contact between the heater 120 and a significant portion of the reservoir cartridge 100 during the treatment of a patient. For example, FIG. 8A illustrates an exemplary reservoir cartridge 100 within a heater 120. As illustrated by FIG. 8A, the reservoir cartridge 100 may be substantially full of fluid and a significant portion of the reservoir cartridge that contains fluid may be in contact with the heater 120. This may ensure that the fluid within the reservoir cartridge 100 is adequately heated.

FIG. 8B illustrates an exemplary reservoir cartridge within a heater during the treatment of a patient. As illustrated by FIG. 8B, the configuration of the second portion 710 of the heater 120 may allow a significant portion of the reservoir cartridge 100 containing fluid to remain in contact with the heater even as fluid is removed from the cartridge during treatment. As illustrated by FIG. 8B, the angle of the second portion 710 of the heater may force fluid within the reservoir cartridge 100 toward the portion of the reservoir cartridge in contact with the first 705 and/or second portions 710 of the heater 120. This may help the fluid within the reservoir cartridge 100 remain adequately heated during treatment.

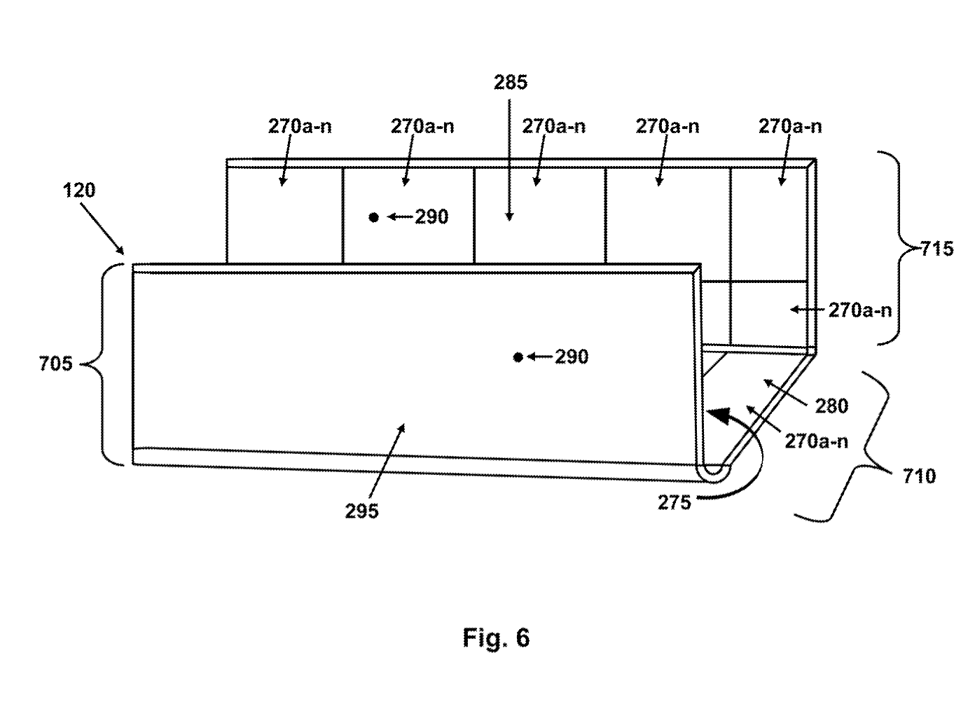

In an embodiment, heater 120 may have multiple heater elements. FIG. 6 illustrates an exemplary heater having heater elements 270a-N according to an embodiment. In an embodiment, a heater element 270a-N may be an independently controllable source of heat. For example, each heater element may be independently controllable from other heater elements. Heater elements 270a-N may be distributed over at least a portion of one or more surfaces of the heater 120. For example, heater elements 270a-N may be organized in rows, columns, diagonally and/or otherwise spaced over at least a portion of a surface of the heater 120. In an embodiment, the heater elements 270a-N may be evenly spaced over at least a portion of a surface of the heater 120.

As described above, a heater 120 may have a first portion 705, a second portion 710 and a third portion 715. In an embodiment, the first portion 705, second portion 710 and/or third portion 715 may have an inner surface 275, 280, 285. An inner surface 275, 280, 285 may be a surface located proximate a reservoir cartridge 100 during heating. In an embodiment, heater elements 270a-N may be located on at least a portion of one or more of the inner surfaces 275, 280, 285.

In an embodiment, one or more of the heater elements 270a-N may be associated with one or more temperature sensors 290, such as a thermistor or the like. In an embodiment, one or more temperature sensors 290 may be located on the surface 275, 280, 285 of its corresponding heater element 270a-N. In an embodiment, one or more temperature sensors 290 may be located on an outer surface of a portion of the heater 120. A temperature sensor 290 located on an outer surface may correspond to one or more heater elements 270a-N on the inner surface of that portion. For example, FIG. 6 illustrates a temperature sensor 290 located on the outer surface 295 of the first portion 705. This temperature sensor 290 may correspond to one or more heater elements 270a-N located on the inner surface 275 of the first portion 705.

A temperature sensor 290 may be connected to a heater element and the computer 125 via one or more wires. The wires may facilitate communication between the heater elements 270a-N and the computing device 125. In an alternate embodiment, one or more temperature sensors 290 may communicate with the hyperthermia apparatus wirelessly.

In an embodiment, the heat flow of each heater element 270a-N may be independently controlled. For example, the heat flow to a heater element 270a-N may be manually controlled or controlled by a computer 125. In an embodiment, the temperature sensor 290 may measure the temperature of the portion of the reservoir cartridge 100 that is in contact with an associated heater element 270a-N.

In an embodiment, computer 125 may be an integrated computer, meaning that the computer and visual display are in the same unit. In one embodiment, the visual display may be a touch-screen. In another embodiment, computer 125 may be removable from housing 180. For example, computer 125 may be removed from housing 180 prior to transport of the apparatus, and connected to the apparatus prior to treatment of a patient.

In an embodiment, reservoir cartridge 100 may store or receive fluid 130 to be administered to a patient. Reservoir cartridge 100 may be disposable, and may have an inlet 140 and an outlet 135. In an embodiment, reservoir cartridge 100 may comprise a lock out circuit, a resistor circuit and/or the like. Lock out circuit may include a fuse link that may be deactivated after the treatment of a patient is completed. For example, a fuse link may short circuit when treatment is completed. This may prevent unauthorized use of and/or unauthorized re-use of disposable components, such as reservoir cartridge 100. Reservoir cartridge 100 may comprise one or more inlets and/or outlets. The inlets and/or outlets may be sealed to prevent the escape of fluid 130, and may facilitate the maintenance of a sterile environment when reservoir cartridge 100 is not connected to the hyperthermia apparatus.

In an embodiment, fluid 130 may be contained in reservoir cartridge 100. Fluid 130 may be introduced into reservoir cartridge 100 via fluid introduction tube 185. Fluid introduction tube 185 may include one or more valves, clamps, or inlets to allow one to introduce a physiologically compatible solution such as a drug into fluid 130 at a controlled rate. Such devices are known in the art and include, for example, IV spikes 190, 195.

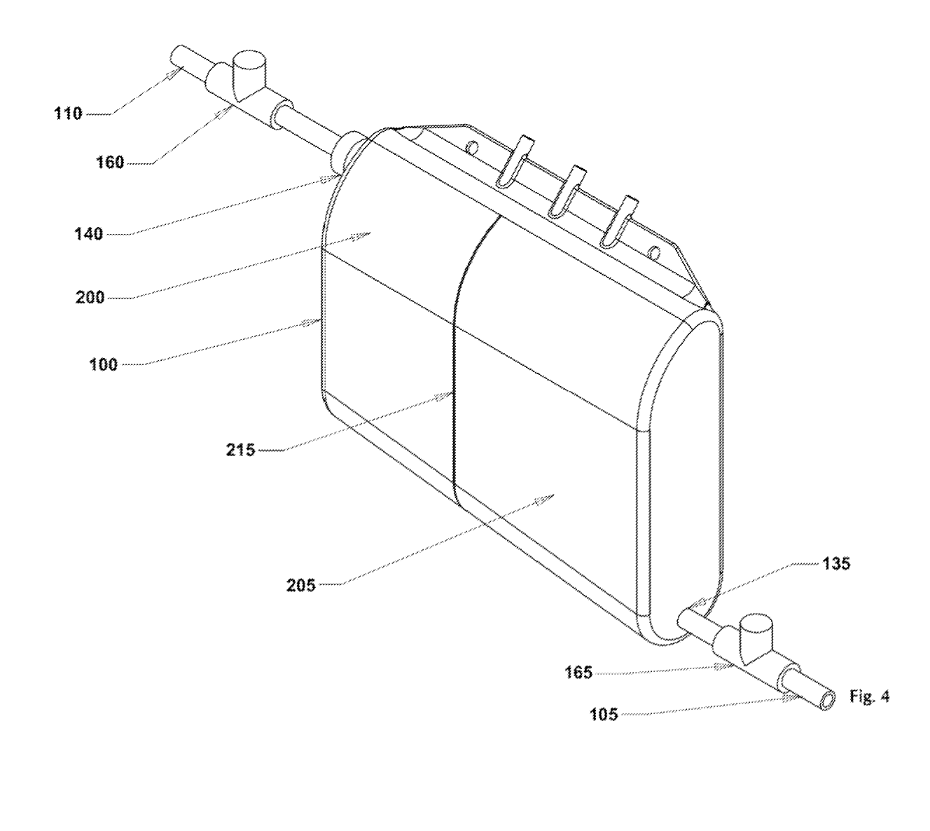

In an embodiment, reservoir cartridge 100 may be fabricated from PVC plastic film and/or other plastic materials. Reservoir cartridge 100 may be RF welded and/or RF heat sealed. In an embodiment, reservoir cartridge 100 may comprise a screen 215. FIG. 4 illustrates an exemplary reservoir cartridge 100 and screen 215. Screen 215 may be located in the proximity of inlet 140. In an embodiment, screen 215 may divide reservoir cartridge 100 into an inflow chamber 200 and an outflow chamber 205. Inflow chamber 200 may receive fluid 130 from inflow tube 110. Fluid 130 may pass through screen 215 into second chamber 205. Screen 215 may filter macroscopic residue, such as fatty tissue, from fluid 130 returning to reservoir cartridge 100 via inflow tube 110. Screen 215 may be fabricated from plastic and/or the like and may be disposable. Screen 215 may be able to filter particles having a size of about 100-140 microns or larger.

In an embodiment, fluid 130 comprises a sterile fluid. In another embodiment, fluid 130 comprises drugs, medication or the like. In an embodiment, hyperthermia assists to render a chemotherapeutic agent more effective against a target disease than the agent would be without the use of hyperthermia. In an embodiment, fluid 130 may comprise one or more chemotherapeutic agents such as cyclophosphamide, doxorubicin, melphalan, mitomycin C, cisplatin, gemcitabine, mitoxantrone, oxaliplatin, etoposide, irinotecan, paclitaxel, docetaxel, 5-Fluorouracil, floxuridine, carboplatin, or other chemotherapeutic agents as would be well-known by one of skill in the art.

In an embodiment, reservoir cartridge 100 and fluid 130 may be heated by heater 120. In an embodiment, different portions of the reservoir cartridge 100 may be heated by one or more heater elements 270a-N. In an embodiment, the temperature sensor 290 of one or more heating elements 270a-N may measure the temperature of the portion of the reservoir cartridge 100 that the heating element contacts. As such, the temperature sensor 290 may measure the temperature of the fluid in the reservoir cartridge 100 at that location.

In an embodiment, the temperature sensors 290 may allow monitoring of the temperature of the reservoir cartridge 100 at certain locations. The temperature sensors 290 may measure the temperature of the portion of the reservoir cartridge 100 in contact with the temperature sensor. In an embodiment, the temperature sensors 290 may be in communication with the computer 125 and may transmit the measured temperatures to the computer.

In an embodiment, the computer 125 may monitor the temperature readings received from the temperature sensors 290, and may control the heat flow to the heating elements 270a-N based on the received temperature readings. For example, the computer 125 may receive a temperature reading from a temperature sensor 290 that it determines to be too high. For example, the computer 125 may compare a received temperature reading to a threshold temperature value. If the received value exceeds a threshold value, the computer 125 may determine that the measured temperature is too high. In response, the computer 125 may reduce or eliminate current to that specific heating element 270a-N.

Similarly, the computer 125 may receive a temperature reading from a temperature sensor 290 that it determines is too low. For example, the computer 125 may compare a received temperature reading to a threshold temperature value. If the received value is less than a threshold value, the computer 125 may determine the measured temperature is too low. In response, the computer 125 may increase current to that specific heating element 270a-N.

In an embodiment, the heater 120 may be utilized to maximize heat transfer, so the apparatus' power consumption may be up to about 15 amps. Alternatively, other power consumption values may be up to about 30 amps.

In an embodiment, once fluid 130 reaches a desired temperature, it may be pumped through outflow tube 105 to patient 150 via pump 115. In an embodiment, outflow tube 105 is disposable, with a proximal end and a distal end. Outflow tube 105 is connected at its proximal end (reservoir cartridge end) to reservoir cartridge 100 at outlet 135, while the distal end (outflow catheter end) of outflow tube 105 is connected to outflow catheter 145. Outflow catheter 145 may be inserted into a patient 150.

In an embodiment, pump 115 may be a paddle wheel, a roller pump, a pulsatile pump, centrifugal pump and/or the like. In an embodiment, pump 115 is in contact with outflow tube 105, and pumps fluid 130 at a rate of up to about 4,000 ml per minute. The high flow rate as compared to prior devices may be critical in providing beneficial treatment by maximizing contact of fluid with a patient. In addition, a high flow rate maintains the temperature of the fluid, which is an important feature of effective hyperthermia. The flow rate in combination with heat provided by a fluid may thereby increase the efficacy of a hyperthermia treatment.

In an embodiment, fluid 130 may be administered to a patient 150, and may be re-circulated to reservoir cartridge 100 through inflow tube 110. Re-circulation of the heated fluid may be used to elevate a patient's core temperature and/or to maintain an elevated temperature for a period of time.

In an embodiment, inflow tube 110 is disposable, with proximal and distal ends. Inflow tube 110 may be connected at its distal end (inflow catheter end) to a patient 150 via inflow catheter 155, and may be connected at its proximal end (reservoir cartridge end) to reservoir cartridge 100 at inlet 140. Fluid 130 coming from patient 150 may be re-heated in reservoir cartridge 100, and once again pumped to a patient 150. This process may continue for a specified period of time with multiple cycles of re-circulation, as may be desired for a given therapeutic effect.

In an embodiment, one or more of inflow tube 110 and outflow tube 105 may comprise a flange or other similar portion. Inflow tube 110 may have a flange at its distal end (inflow catheter end) and outflow tube 105 may have a flange at its distal end (outflow catheter end). The flange may be fabricated from plastic and/or any other suitable material. In an embodiment, the flange may assist a physician or other healthcare professional to more quickly and efficiently suture the inflow tube 110 and/or the outflow tube 105 to the patient 150. Moreover, the flange may serve as a seal for the distal end (inflow catheter end) of the inflow tube 110 and/or the distal end (outflow catheter end) of the outflow tube 105.

According to an embodiment, the hyperthermia apparatus of the invention includes sensors for monitoring the temperature and pressure of the fluid. In an embodiment, a temperature sensor may be a standard thermistor, an infrared thermistor or the like. As illustrated in FIG. 1, temperature sensors 160, 165 may be located in reservoir cartridge 100 at the proximal ends of inflow tube 110 and outflow tube 105. Temperature sensors 160, 165 may allow monitoring of a fluid's 130 temperature as it both enters and leaves reservoir cartridge 100. In an embodiment, temperature sensors 160, 165 are disposable. In an embodiment, temperature sensors 160, 165 are in communication with a computer 125.

In an embodiment, a system for implementing hyperthermia may include one or more auxiliary temperature sensors and a hyperthermia apparatus such as that described in this disclosure. The auxiliary temperature sensors may be placed on and/or in the patient at various locations. One or more auxiliary temperature sensors may be plug-in thermistors that may be connected to the hyperthermia apparatus via a standard connection or the like. In another embodiment, one or more auxiliary temperature sensors may communicate with the hyperthermia apparatus wirelessly. In an embodiment, a healthcare professional may select one of the auxiliary temperature sensors to use as a reference. The healthcare professional may be able to monitor the temperature at the location of the auxiliary temperature sensors, and the hyperthermia apparatus may control the temperature of the fluid based on the selected auxiliary temperature sensor rather than the temperature sensors 160, 165 located in the hyperthermia apparatus.

As illustrated in FIG. 1, pressure sensor 175 is located, according to an embodiment, in outflow tube 105 near reservoir cartridge 100. Preferably, pressure sensor 175 is located within outflow tube 105 downstream from pump 115. In an embodiment, pressure sensor 175 is located within outflow tube 105 immediately downstream from pump 115. Pressure sensor 175 may allow monitoring of fluid's 130 pressure as fluid 130 leaves reservoir cartridge 100. In an embodiment, pressure sensor 175 is in communication with computer 125. In an embodiment, pressure sensor 175 is disposable.

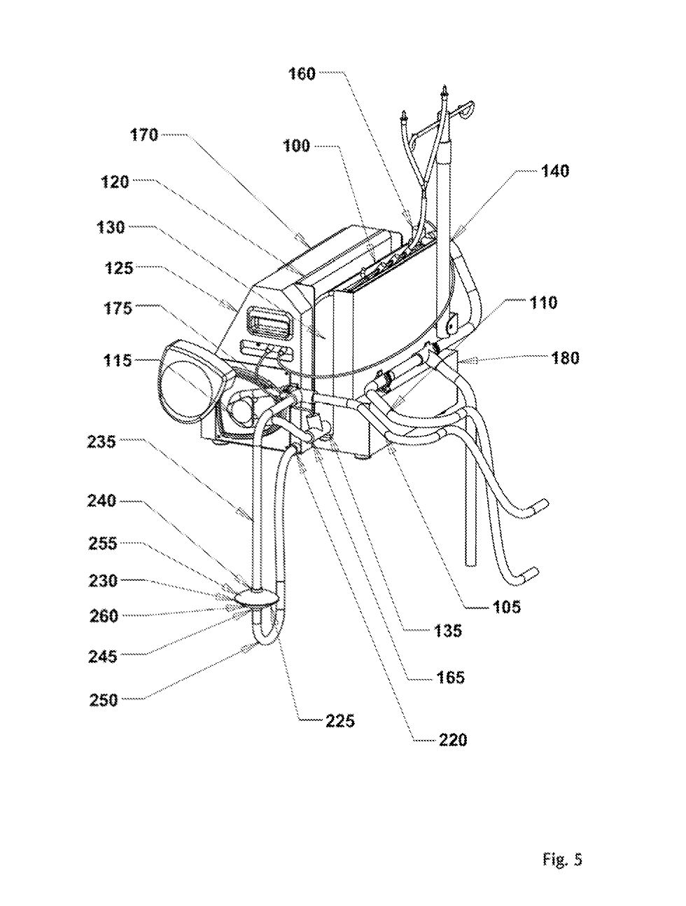

In another embodiment, housing may contain a pressure sensor 220. Pressure sensor 220 may be durable. Pressure sensor 220 may measure pressure of fluid 130 via a pressure isolator 225. As illustrated by FIG. 5, pressure isolator 225 may comprise a first chamber 255, a second chamber 260, an inlet 240, an outlet 245 and/or a membrane 230. A first connector tube 235 may connect outflow tube 105 to inlet 240. A second connector tube 250 may connect outlet 245 to pressure sensor 220. Membrane 230 may separate first chamber 255 from second chamber 255. Membrane 230 may be fluid impermeable and may prevent fluid 130 from coming into contact with pressure sensor 220. In an embodiment, pressure sensor 220 may measure the pressure of fluid 130 based on a pressure differential between first chamber 255 and second chamber 260.

Although the figures illustrates specific placements of temperature sensors 160, 165 and pressure sensors 175, 220 it is understood that sensors 160, 165, 175, 220 may be placed in different locations on the apparatus. In addition, one or more of temperature sensors 160, 165 and pressure sensors 175, 220 may wirelessly communicate with computer 125. Moreover, additional temperature and/or pressure sensors may be implemented by the device of the invention.

The apparatus of the invention may be used to monitor a fluid's 130 temperature while the fluid is in reservoir cartridge 100, which may allow for accurate temperature control to within about plus or minus one-half of one degree Centigrade (0.5° C.). In other words, the temperature variation of a fluid from when it leaves reservoir cartridge 100 to when it enters a patient 150 may be, for example, about 0.5° C. or less. In a preferred embodiment, the temperature of fluid 130 in reservoir cartridge 100 does not exceed about 43° C. As such, the temperature of fluid 130 when administered to a patient 150 preferably does not exceed about 42.5° C. The temperature of a fluid 130 in reservoir cartridge 100 may be maintained at a temperature other than 43° C., for example, any temperature desired by an operator to achieve the desired therapeutic effect, or for operation of the device in prime mode, as described below.

Computer 125 and touch screen 170 are, in one embodiment, configured to provide audible and visible alarms if certain conditions occur. For example, if the temperature of a fluid 130 in reservoir cartridge 100 exceeds a specified temperature, a visible and/or audible alarm may be triggered. Likewise, if the pressure or temperature of fluid 130 exceeds a preset threshold, a visible and/or audible alarm may be triggered.

As a safety precaution, heater 120, according to one embodiment, stops providing heat if a fluid's 130 temperature exceeds a specified temperature. In an embodiment, heater 120 stops providing heat if fluid's 130 pressure exceeds a specified level.

Similarly, pump 115, in an embodiment, stops pumping fluid 130 if the fluid's 130 pressure exceeds a specified level. In an embodiment, pump 115 stops pumping fluid 130 if fluid's 130 temperature exceeds a specified level.

Computer 125 may include a processor and a processor-readable storage medium. Computer 125 is programmable and capable of receiving input from a user. For example, a user may specify temperature levels, pressure levels or the like via the touch screen 170 or other input interfaces. A user may also input other information, such as the duration of the treatment, the amount of time the apparatus is to operate in prime mode or the like. In an embodiment, computer 125 may record data such as measurements associated with treatment and the like. For example, during the treatment of a patient, computer 125 may record one or more temperatures at one or more temperature sensors 160, 165, auxiliary temperature sensors and/or the like. Computer 125 may also record flow rates, pressure values, treatment time and/or the like.

Computer 125 is in communication with pump 115, heater 120, temperature sensors 160, 165, and pressure sensors 175, 220. Computer 125 controls the operation of pump 115 and monitors temperature sensors 160, 165 and pressure sensors 175, 220. In an embodiment, if the temperature or pressure of fluid 130 exceeds a specified level, computer 125 provides audible and visual alarm signals. In another embodiment, computer 125 shuts off heater 120 if fluid's 130 temperature exceeds a specified temperature or if fluid's 130 temperature is outside a specified range of temperatures. Likewise, in an embodiment, computer 125 shuts off pump 115 if fluid's 130 pressure exceeds a specified pressure level or if fluid's 130 pressure is outside a specified range of pressure levels. Computer 125 may shut off pump 115 if fluid's 130 temperature exceeds a specified temperature or if fluid's 130 temperature is outside a specified range of temperatures. Similarly, computer 125 may shut off heater 120 if fluid's 130 pressure level exceeds a specified pressure level or if fluid's 130 pressure level is outside a specified range of pressure levels.

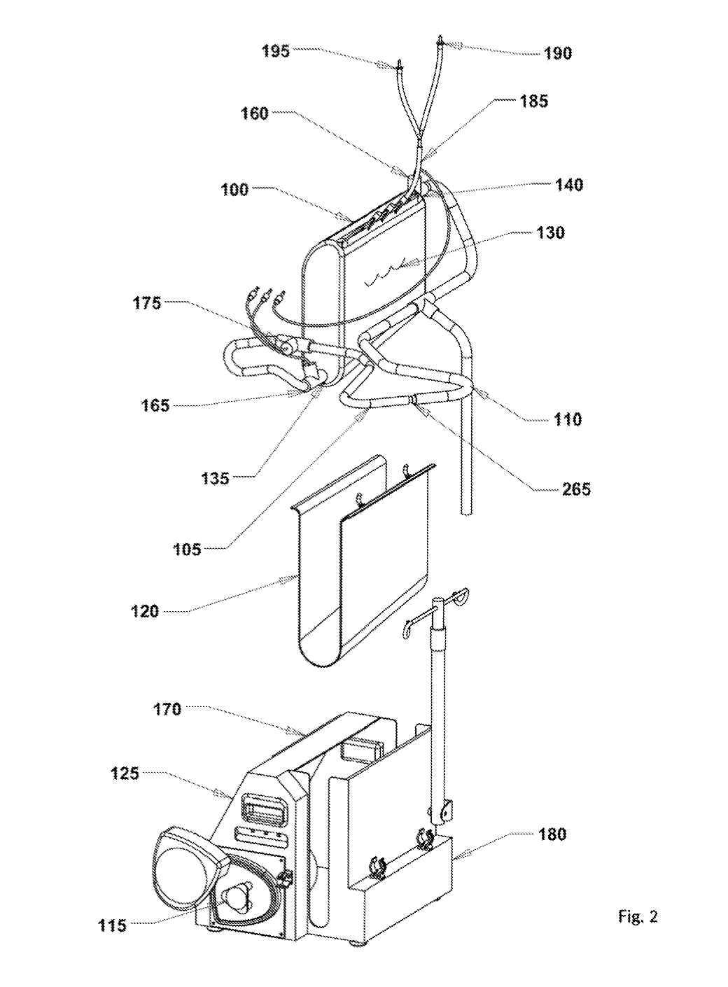

In an alternate embodiment, the apparatus of the invention operates without being connected to a patient. This is referred to as “prime mode” and is illustrated in FIG. 2. In prime mode, the apparatus prepares fluid 130 to be administered to a patient by heating and re-circulating fluid 130. In prime mode, outflow tube 105 may be connected to the inflow tube 110 via connector 265. A variety of tubing connectors suitable for use in the invention are known in the art and may include, for example, a barbed tubing connector or the like, or other connector as may be suitable to achieve the desired connector function. As such, the fluid 130 may be pumped from reservoir cartridge 100 through outflow tube 105 through connector 265 and back to reservoir cartridge 100 through inflow tube 110. Fluid 130 may be pumped for a specified period of time before fluid 130 is administered to a patient. When operating in prime mode, the temperature of fluid 130 in the reservoir may be maintained at a temperature up to, for example, 53° C. The device of the invention may be run in prime mode to ensure that the temperature of fluid 130 does not drop below an acceptable level before the apparatus is connected to a patient. An operator may set a time period for the apparatus to operate in prime mode.

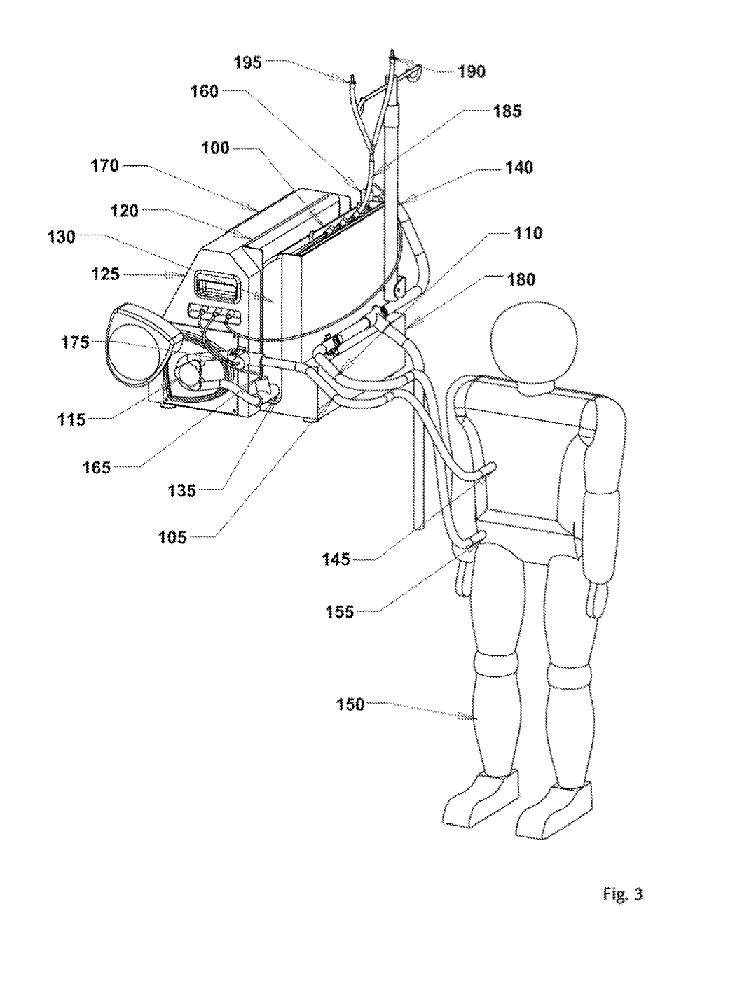

In an embodiment, reservoir cartridge 100, heater 120 and computer 125 are contained in housing 180 (FIG. 3). In one embodiment, these elements are located in close proximity to each other. The proximity of elements contribute to the apparatus' portability and ease of use in a variety of clinical settings, including both inside and outside of an operating room.

It will be appreciated that various of the above-disclosed and other features and functions, or alternatives thereof, may be desirably combined into many other different systems or applications. Various presently unforeseen or unanticipated alternatives, modifications, variations or improvements therein may be subsequently made by those skilled in the art which are also intended to be encompassed by the following claims.

What is claimed is:

1. A heating system for a hyperthermia apparatus, the system comprising:
a heater comprising:
a plurality of independently controllable heater elements located on one or more surfaces of the heater, wherein each of the surfaces is proximate to a reservoir cartridge, and
a temperature sensor associated with one or more of the heater elements; and
a computing device in communication with the temperature sensor, wherein the computing device is configured to adjust an amount of electrical current to the one or more heater elements associated with the temperature sensor based on a temperature reading received from the temperature sensor.

2. The heating system of claim 1, wherein the heater comprises:
a first portion comprising a first inner surface;
a second portion comprising a second inner surface; and
a third portion comprising a third inner surface,
wherein the first portion is connected to the second portion,
wherein the third portion is connected to the second portion,
wherein the first inner surface is located opposite the third inner surface,
wherein the heater elements are located on one or more of the first inner surface, the second inner surface and the third inner surface.

3. The heating system of claim 1, wherein the heater comprises:
a first portion;
a second portion; and
a third portion,
wherein the first portion and the third portion are connected to the second portion,
wherein the second portion is located at an angle relative to one or more of the first portion and the third portion.

4. The heating system of claim 1, wherein the heater elements are arranged in one or more of following ways on the one or more surfaces of the heater:
in one or more rows;
in one or more column; and
diagonally.

5. The heating system of claim 1, wherein the heater elements are evenly spaced on the one or more surfaces of the heater.

6. The heating system of claim 1, wherein the computing device is configured to:
compare a temperature reading received from the temperature sensor to a threshold value; and
in response to the received temperature reading exceeding a threshold value, decrease an amount of current to at least one of the heater elements associated with the temperature sensor.

7. The heating system of claim 1, wherein the computing device is configured to:
compare a temperature reading received from the temperature sensor to a threshold value; and
in response to the received temperature reading not exceeding a second threshold value, increase an amount of current to at least one of the heater elements associated with the temperature sensor.

8. The heating system of claim 1, wherein the computing device is configured to:
compare a temperature reading received from the temperature sensor to a range of values; and
in response to the received temperature reading falling outside of the range of values, decrease an amount of current to at least one of the heater elements associated with the temperature sensor.

9. The heating system of claim 1, wherein the computing device is configured to:
compare a temperature reading received from the temperature sensor to a range of values; and
in response to the received temperature reading falling outside of the range of values, increase an amount of current to at least one of the heater elements associated with the temperature sensor.

10. The heating system of claim 1, wherein the heating system is contained within an apparatus for performing hyperthermia.

11. The heating system of claim 1, wherein the temperature sensor is configured to communicate wirelessly with the computing device.

12. The heating system of claim 1, wherein the temperature sensor is configured to communicate with the computing device via a wired connection.

13. A heating system for a hyperthermia apparatus, the system comprising:
a heater comprising:
a first portion having a first surface,
a second portion having a second surface,
a third portion having a third surface,
a plurality of independently controllable heater elements located on one or more of the first surface, the second surface and the third surface, and
a temperature sensor associated with one or more of the heater elements; and
a computing device in communication with the temperature sensor, wherein the computing device is configured to adjust an amount of electrical current to one or more heater elements associated with the temperature sensor based on a temperature reading received from the temperature sensor.

14. The heating system of claim 13, wherein the second portion is positioned at an angle relative to one or more of the first portion and the third portion.

15. The heating system of claim 13, wherein the heater elements are evenly spaced on one or more of the first surface, the second surface and the third surface.

16. The heating system of claim 13, wherein the computing device is configured to:
compare a temperature reading received from the temperature sensor to a threshold value; and
in response to the received temperature reading exceeding a threshold value, decrease an amount of current to at least one of the heater elements associated with the temperature sensor.

17. The heating system of claim 13, wherein the computing device is configured to:
compare a temperature reading received from the temperature sensor to a threshold value; and
in response to the received temperature reading not exceeding a second threshold value, increase an amount of current to at least one of the heater elements associated with the temperature sensor.

18. The heating system of claim 13, wherein the computing device is configured to:
compare a temperature reading received from the temperature sensor to a range of values; and
in response to the received temperature reading falling outside of the range of values, decrease an amount of current to at least one of the heater elements associated with the temperature sensor.

19. The heating system of claim 13, wherein the computing device is configured to:
compare a temperature reading received from the temperature sensor to a range of values; and
in response to the received temperature reading falling outside of the range of values, increase an amount of current to at least one of the heater elements associated with the temperature sensor.

20. The heating system of claim 13, wherein at least one of the one or more temperature sensors is located on an inner surface of one or more of the first portion, the second portion and the third portion.
